(19) United States
(12) Patent Application Publication
(10) Pub. No.: US 2021/0068736 A1
(43) Pub. Date: Mar. 11, 2021

(54) METHOD AND DEVICE FOR SENSING PHYSIOLOGICAL STRESS

(71) Applicant: Sony Corporation, Tokyo (JP)

(72) Inventors: Torbjörn GRAHM, MALMÖ (SE); Olof ZANDER, SÖDRA SANDBY (SE); Anders EDENBRANDT, LUND (SE); Mazloum NAFISEH, LUND (SE)

(21) Appl. No.: 16/960,141

(22) PCT Filed: Jan. 29, 2019
(86) PCT No.: PCT/SE2019/050068

§ 371 Date: Jul. 6, 2020

(30) Foreign Application Priority Data
Feb. 2, 2018 (SE) 1850122-1

(51) Int. Cl. A61B 5/16; A61B 5/0205; A61B 5/029; A61B 5/1455; A61B 5/145; A61B 5/11; A61B 5/00; G16H 40/67; G16H 50/30; G16H 50/70

(52) U.S. Cl. CPC A61B 5/165; A61B 5/02055; A61B 5/029; A61B 5/14551; A61B 5/14546; A61B 5/163; A61B 5/021; A61B 5/6801; A61B 5/6898; G16H 40/67; G16H 50/30; G16H 50/70; A61B 5/1107

(57) ABSTRACT

A monitoring device or arrangement performs a method (30) of sensing physiological stress in a user based on sensor data from one or more sensors associated with the user. The method comprises: obtaining (31) stress parameter values (SPVs) that represent a physiological stress level in the user and that are generated at a measurement rate based on the sensor data; monitoring (33) the stress parameter values (SPVs) for detection of a switching point, which is detected when the stress parameter values indicate an elevated stress level in the user for a predefined time period; and causing (34) a reduction of the measurement rate upon detection of the switching point.

### TECHNICAL FIELD

The present invention relates generally to monitoring of physiological stress in a user based on data generated by one or more sensors.

### BACKGROUND

In today's society, the impact of physiological stress on the human body is gaining more and more attention. Stress is the body's method of reacting to a challenge and is controlled by multiple systems in the body. The autonomic nervous system (ANS) and the so-called hypothalamic-pituitary-adrenal (HPA) axis form major parts of the body's stress system. The ANS responds reflexively to both physical stressors and to higher level inputs from the brain. The ANS is composed of the sympathetic and parasympathetic nervous systems, two branches that are both tonically active with opposing activities. The activity of the sympathetic nervous system drives what is called the “fight or flight” response. The HPA axis is a neuroendocrine system that mediates a stress response by the sympathetic nervous system, e.g. by releasing the hormones adrenaline (also known as epinephrine) and cortisol into the bloodstream. Adrenaline is released first to prepare the body for action and to trigger the fight or flight response. Cortisol is released later and over a longer time and helps maintain fluid balance and blood pressure. A human that is subjected to excessive physiological stress will build up high and persistent levels of cortisol, which is may lead to serious health issues.

Finding ways to effectively measure stress is an important area of research, as it is the first step in recognizing and potentially reducing stress, therefore minimizing the adverse mental and physical effects of stress. There are several existing methods to measure stress, ranging from invasive methods such as analyzing cortisol levels in saliva or blood, to perceptive methods such as administering multi-item questionnaires. Recently, smartphone applications and wearable devices have emerged that operate to estimate the stress level in the user based on data from one or more sensors associated with the user, e.g. a heart rate monitor.

The prior art comprises US2005/0154264 which discloses a data processing system for monitoring stress levels of a user and providing remedial feedback in response to elevated levels of more or more physiological indicators measured by one or more physiologic sensors worn by the user.

US2014/0135612 discloses a biometric monitoring device for attachment to a body part of a user and including sensor(s) for capturing physiological conditions of the user. In one embodiment, the monitoring device is configured to increase the sampling rate and/or the sampling resolution mode of sensors employed to acquire heart rate data when the amount of user motion indicated by a motion sensor exceeds a threshold. In another embodiment, the monitoring device is configured to operate sensors in a higher-rate sampling mode and calculate heart rate variability (HRV) when the device determines that the user is sedentary or asleep. The device may employ HRV as an indicator of cardiac health or stress.

Further, US2017/0071523 discloses a method of monitoring the stress level of a user based on biological measurements of one or more physiological markers. In one embodiment, the method dynamically switches from low-power mode to high-power mode when one or more of the physiological markers are above a threshold level. The high-power mode may involve a longer detection time period and a higher frequency of detection time periods compared to the low-power mode.

There is a continued need to optimize the performance of stress monitoring, e.g. with respect to power consumption and processing efficiency.

### SUMMARY

It is an objective of the invention to at least partly overcome one or more limitations of the prior art.

Another objective is to enable resource-efficient monitoring of an individual's physiological stress level.

One or more of these objectives, as well as further objectives that may appear from the description below, are at least partly achieved by a method, a computer-readable medium, a device, and an arrangement according to the independent claims, embodiments thereof being defined by the dependent claims.

A first aspect of the invention is a method of sensing stress in a user. The method comprises: obtaining stress parameter values that represent a physiological stress level in the user and that are generated at a measurement rate based on sensor data from one or more sensors associated with the user; monitoring the stress parameter values for detection of a switching point, wherein the switching point is detected when the stress parameter values indicate an elevated stress level in the user for a predefined time period; and causing a reduction of the measurement rate upon detection of the switching point.

In some embodiments, the method further comprises: causing the at least one sensor to be selectively activated and inactivated to generate the sensor data in coordination with the measurement rate.

In some embodiments, the predefined time period is in the range of 15-60 minutes, and preferably in the range of 20-45 minutes.

In some embodiments, the predefined time period corresponds to a time period in which the physiological stress in the user is at least partly controlled by adrenaline.

In some embodiments, the measurement rate is selected so that at least two, and preferably at least three, stress parameter values are obtained within the predefined time period.

In some embodiments, the switching point is detected when a predefined number of consecutive stress parameter values exceeds a stress threshold value.

In some embodiments, the method further comprises, subsequent to the switching point, monitoring the stress parameter values for detection of a further switching point, and causing an increase in the measurement rate upon detection of the further switching point.

In some embodiments, the further switching point is detected when one or more of the stress parameter values fall below a stress threshold value within a further predefined time period subsequent to the switching point, or upon expiry of the further predefined time period.

In some embodiments, the further predefined time period is in the range of 4-10 hours.

In some embodiments, the method further comprises: obtaining the sensor data from the one or more sensors, and generating the stress parameter values based the sensor data.

In some embodiments, the sensor data represents one or more of heart activity, time interval between consecutive heart beats, heart rate, heart rate variability, skin conductivity, skin temperature, blood pressure, pupil diameter, eye movement, breathing pattern, blood oxygen saturation level, cardiac output, peripheral blood flow, muscle tension, and hormone content.

A second aspect of the invention is a computer-readable medium comprising computer instructions which, when executed by a processor, cause the processor to perform the method of the first aspect or any of its embodiments.

A third aspect of the invention is a device for sensing stress in a user. The device is configured to: obtain stress parameter values that represent a physiological stress level in the user and are generated based on sensor data from one or more sensors associated with the user; monitor the stress parameter values for detection of a switching point when the stress parameter values indicate an elevated stress level in the user for a predefined time period; and cause a reduction of the measurement rate upon detection of the switching point.

In some embodiments, the device further comprises at least one of said one or more sensors.

In some embodiments, the device is a mobile communication device, a portable electronic device or a wearable computer.

A fourth aspect of the invention is an arrangement for sensing stress in a user. The arrangement comprises: one or more sensors associated with the user; a first module configured to generate, at a measurement rate and based on data sensor data generated by the one or more sensors, stress parameter values that represent physiological stress in the user; and a second module configured to monitor the stress parameter values for detection of a switching point when the stress parameter values indicate an elevated stress level in the user for a predefined time period and to reduce the measurement rate upon detection of the switching point.

Any one of the above-identified embodiments of the first aspect may be adapted and implemented as an embodiment of the second to fourth aspects.

Other objectives, as well as features, aspects and advantages of embodiments of the present invention will appear from the following detailed description, from the attached claims as well as from the drawings.

### BRIEF DESCRIPTION OF DRAWINGS

FIG. 1 is a block diagram of a system environment for stress monitoring.

FIG. 2 schematically illustrates the timing of adrenaline and cortisol release by a stress system in a human.

FIG. 3 is a flow chart of a method for sensing stress in accordance with an embodiment.

FIG. 4 shows an example of data gathering and computation of stress parameter values in accordance with an embodiment of the invention.

FIG. 5 is a block diagram of an arrangement for sensing stress in accordance with an embodiment.

FIGS. 6A-6D are block diagrams of example implementations of the arrangement in FIG. 5.

FIG. 7 is a flow chart of a method for sensing stress in accordance with an embodiment.

FIG. 8 is a block diagram of a device for sensing stress in accordance with an embodiment.

### DETAILED DESCRIPTION OF EXAMPLE EMBODIMENTS

Embodiments of the present invention will now be described more fully hereinafter with reference to the accompanying schematic drawings, in which some, but not all, embodiments of the invention are shown. Indeed, the invention may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments of the present invention described and/or contemplated herein may be included in any of the other embodiments of the present invention described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more,” even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention.

Well-known functions or constructions may not be described in detail for brevity and/or clarity. Unless otherwise defined, all terms (including technical and scientific terms) used herein have the same meaning as commonly understood by one of ordinary skill in the art to which this invention belongs.

Before describing embodiments of the invention in more detail, a few definitions will be given.

As used herein, “physiological stress” (also known as “biological stress”) refers generally to a state of heightened level of physiological activity caused by physical, mental, or emotional factors (stressors) that cause bodily or mental tension. Typically, the state of stress involve a body balance in which the overall cardiovascular function, as represented by e.g. heart rate and cardiovascular output, is substantially higher than the level required by immediate physical metabolic requirements. Physiological stress may be due to different sources, e.g. physical load, physical condition, mental stress, low level of resources, or emotional arousal.

As used herein, a “stress parameter value” (abbreviated SPV) is a “stress index” that indicates the level of physiological stress in an individual. The stress index may be an absolute or relative measure and is computed based on sensor data from a sensor associated with an individual or user. Embodiments described herein are not limited to any particular stress index and may be applied to any type of sensor data. Stress detection is an ongoing research topic among both psychologists and engineers. Various techniques have been developed for human stress detection, e.g. using wearable sensors and bio-signal processing. In a few non-limiting examples, a stress index may be computed based on measurement(s) of blood pressure, heart activity, pupil diameter, eye movement, skin conductivity, skin temperature, breathing pattern, blood oxygen saturation level, cardiac output, peripheral blood flow, muscle tension, hormone content (e.g. amylase and/or cortisol) in blood or saliva, etc. A stress index based on heart activity may be computed based on heart rate (HR), time intervals between consecutive heart beats, and heart rate variability (HRV), or any combination thereof. For example, U.S. Pat. No. 7,330,752 discloses various algorithms for computing a stress index based on measured heart activity, specifically by quantifying HRV, e.g. in terms of spectral power in a low-frequency (LF) and high-frequency (HF) region, respectively. Further examples of stress index based on HR, respiration and HRV are given in “COGCAM: Contact-free Measurement of Cognitive Stress During Computer Tasks with a Digital Camera”, by McDuff et al, Conference paper, CHI'16, May 2016. The use of pupil diameter and eye movement is exemplified in “Automatic Stress Classification With Pupil Diameter Analysis”, by Pedrotti et al, International Journal of Human-Computer Interaction, Vol. 30, No. 3, p. 220-236 (2014), and “Modelling Characteristics of Eye Movement Analysis for stress detection—Performance Analysis using Decision tree approach”, by Kalas et al, International Journal of Engineering and Innovative Technology (IJEIT), Vol., No. 4, p. 20-25 (2017). The use of skin conductance is exemplified in “A Stress Sensor Based on Galvanic Skin Response (GSR) Controlled by ZigBee”, by Villarejo et al, Sensors, Vol. 12, p. 6075-6101 (2012). All of the above-mentioned disclosures are incorporated herein by reference.

It should also be noted that the SPV may be computed based on sensor data from two or more sensors, which may but need not generate different types of sensor data. It is also conceivable that several different SPVs are computed and jointly evaluated for assessing the stress level of the user.

In some embodiments, the SPV may be computed or evaluated for detection of primarily emotionally or mentally induced stress, by excluding the impact of movements, postural changes and other types of physical activity of the monitored user. Examples of such processing is found among the above-mentioned disclosures. The physical activity may be detected based on output data from one or more movement sensors, e.g. accelerometers, and/or any other sensor providing data indicative of physical activity, such as skin temperature, respiration, oxygen consumption, etc.

As used herein, a “sensor” refers to any device that may be associated with a user and is configured to measure and indicate a physical quantity that is related to the user and relevant, in a broad sense, for assessing the level of physiological stress. The sensor data generated by the sensor may be either analog or digital. Non-limiting examples of conceivable sensors include heart activity monitor, sweat sensor, moisture sensor, bio-signal sensor, eye movement sensor, pupil size sensor, blood pressure sensor, peripheral blood flow sensor, pulse oximeter, photoplethysmograph (PPG), skin temperature sensor, muscle tension sensor, saliva analyzer, blood analyzer, insulin sensor, accelerometer, gyroscope, altimeter, pedometer, vibration sensor, ambient temperature sensor, global positioning system (GPS), etc.

A sensor may be “associated with” a user by being worn by the user, e.g. attached to the user's body or clothing or implanted into the user's body, or by being located in proximity of the user in a monitoring situation. In one example, the sensor is included in a wearable or portable device, such as a fitness monitor, a wristband, a chest strap, a helmet, headphone, a mobile phone, an action camera, an adhesive patch, eyeglasses, a hearing aid, etc. In another example, the sensor is installed in the same building or room as the user, in a bed or in an exercise device.

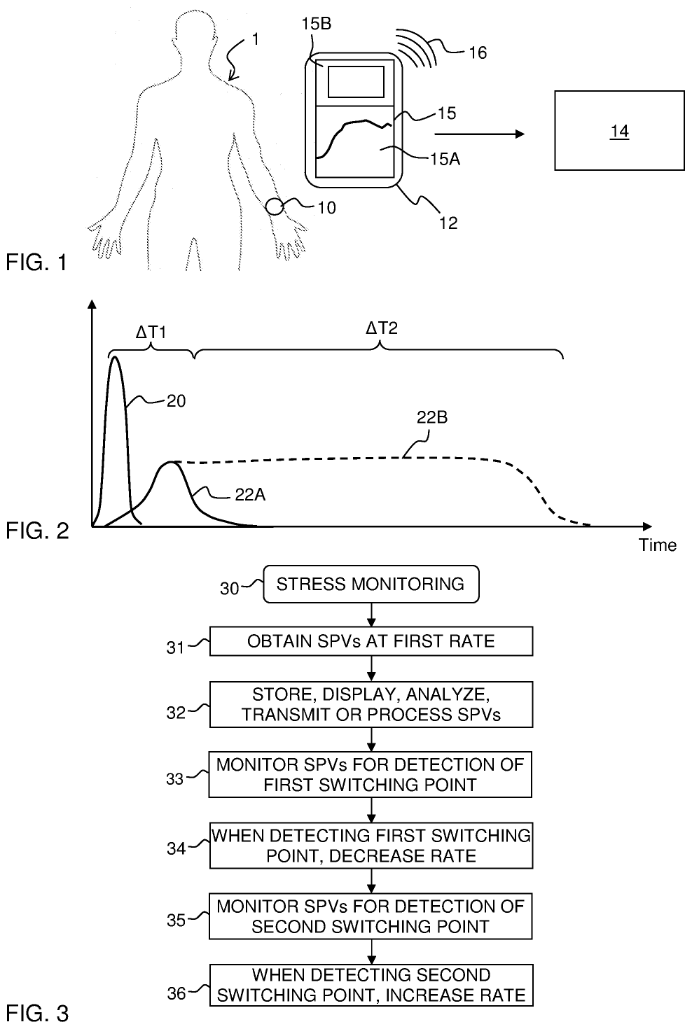

FIG. 1 illustrates an implementation example of stress monitoring in accordance with embodiments of the invention. An individual or user 1 is equipped with a sensor 10. In the illustrated example, sensor 10 is worn on the wrist. The sensor 10 provides sensor data. The sensor data is acquired by a portable electronic device 12, designated PED in the following. The PED 12 may be any electronic device which is capable of being carried, held or worn by a user. For example, the PED 12 may be a handheld device, such as a mobile communication device, tablet, laptop, etc, as well as a wearable computer (“wearable”). The PED 12 may be a generic device capable of performing different tasks, e.g. by executing different application programs, or a specialized device tailored to perform a single specific task.

As will described in greater detail below, the PED 12 is operable as a stress monitoring device that generates, based on the sensor data from the sensor 10, stress parameter values (SPVs) that represent the level of physiological stress in the user 1. As shown, the PED 12 may be further configured to report the SPVs to a computing device 14, which is configured to perform remote stress monitoring, e.g. by storing the SPVs, by displaying the SPVs, by analyzing the SPVs for identification of trends or for prediction, by generating alarms or alerts, etc. Alternatively or additionally, the PED 12 may be configured to perform local stress monitoring, e.g. by storing the SPVs, displaying stress-related data or feedback for the user, or generating an alarm when the SPVs fulfill an alarm criterion. It is also conceivable that the PED 12 or the computing device 14 uses the SPVs to control a device or process that operates on the user 1. For example, such a device or process may control injection of a substance, e.g. a medicament or insulin, into the body of the user 1 based on the current level of stress as indicated by the SPVs.

In the illustrated example, the PED 12 is configured to define a user interface (UI) 15 for displaying SPVs, e.g. in a first UI section or window 15A, in plain text or graphically (as shown). The PED 12 may also display further information in a second UI section or window 15B, e.g. instructions for the user. In the illustrated example, the PED 12 is also operable to selectively generate an alarm signal 16.

FIG. 2 serves to explain the rationale behind embodiments of the present invention and illustrates how adrenalin and cortisol may be released into the bloodstream of a human when its stress system is activated. Curve 20 represents adrenalin concentration and curves 22A, 22B represent cortisol concentration. The curves 20, 22A, 22B are schematic and not drawn to scale in either time or concentration. After exposure to a stressor event, in a first stage, the stress system releases adrenaline into the bloodstream to prepare the body for action. The adrenaline boosts the sympathetic nervous system and causes the individual to experience a faster heart rate, increased sweating, shallower breathing and sharpened senses. The sympathetic activation by adrenaline is fast. In a second stage, the body releases cortisol into the bloodstream. Cortisol release is activated by a chain of other hormones starting from the hypothalamus. Cortisol builds up slowly and also takes a long time to go back to normal. The body's response to a single stressor event may be represented by curves 20, 22A. Although there are individual variations, the physiological impact of a stressor event is typically less than 15-60 minutes, as indicated by a first time period ΔT1 in FIG. 2. However, under certain circumstances, the body may generate a prolonged period of elevated cortisol concentration in the bloodstream, causing the individual to experience an extended period of stress, as indicated by curve 22B. The extended period typically has a duration of several hours, e.g. 4-10 hours, as indicated by second time period ΔT2. In fact, the stress activation is generally bimodal; if the stress does not substantially subside within the first time period ΔT1 it is likely to continue for the second time period ΔT2.

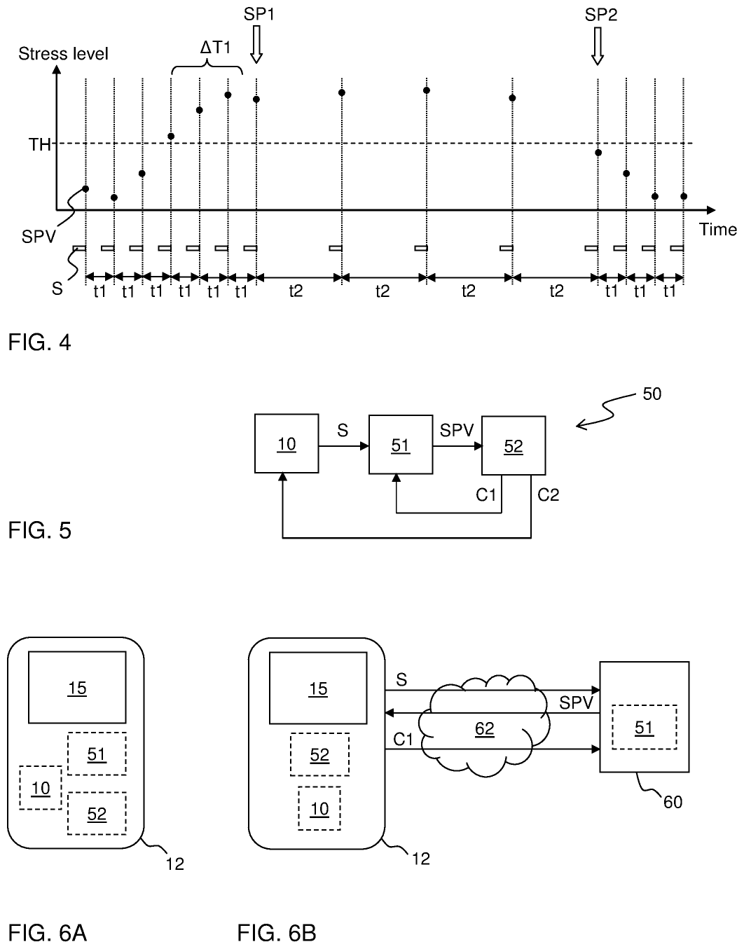

FIG. 3 is a flow chart of a method 30 of monitoring stress in a user in accordance with an embodiment. The method 30 takes advantage of the stress system behavior as discussed in relation to FIG. 2 and may be performed by the PED 12 in relation to the user 1 in FIG. 1. The method 30 produces a time sequence of SPVs, e.g. as exemplified by filled dots in FIG. 4. In step 31, the PED 12 obtains SPVs at a first measurement rate, e.g. given by default settings, based on sensor data from the sensor 10. As will be described further below, the SPVs may but need not be generated by the PED 12. In FIG. 4, the first measurement rate corresponds to a time interval t1 between consecutive SPVs. In step 32, the SPVs may be used for any suitable purpose, e.g. by being stored on the PED 12, displayed on the PED 12, analyzed on the PED 12, transmitted from the PED 12 to the computer device 14, or used by the PED 12 for process control, or any combination thereof. Step 33 monitors the SPVs that are generated for detection of a first switching point. When the first switching point is detected, step 34 causes the measurement rate to be reduced (decreased), which means that the SPVs are obtained by step 31 at longer time intervals. Although not indicated in FIG. 3, the method 30 may also continue to perform step 32 on the SPVs that are generated subsequent to step 34.

In FIG. 4, the first switching point is designated by SP1, and the decreased measurement rate is represented as an increased time interval t2 between consecutive SPVs. As indicated in FIG. 4, the first switching point SP1 is given by a detection criterion that is defined with respect to the first time period ΔT1. Specifically, the first switching point SP1 is detected when the SPVs indicate that user has experienced an elevated stress level for more than the first time period ΔT1. In the example of FIG. 4, an elevated stress level is indicated when an SPV exceeds a predefined threshold TH. The use of SP1 capitalizes on the insight that an elevated stress level tends to either subside within ΔT1, which is relatively short, or persist for ΔT2, which is relatively long, as indicated in FIG. 2. It is thus possible to reduce the measurement rate at SP1 without any major loss of information about the stress level in the user. By reducing the measurement rate at SP1, the method 30 both improves processing efficiency and reduces power consumption. Although not shown in FIG. 3, step 34 may also control the activation of the sensor 10, so that the sensor 10 is selectively activated and inactivated to generate sensor data in coordination with the measurement rate. In other words, the sensor 10 is only activated when sensor data is needed for computing the respective SPV. FIG. 4 indicates, by a sequence of rectangles, the timing of sensor data S for the respective SPV. As shown, the time interval between sensor data S matches the time interval between SPVs. The selective activation of the sensor 10 may lead to considerable power savings, by reducing the duty cycle of the sensor 10. Depending on implementation and type of SPV, the sensor data S may be gathered at a single time point or over an extended data collection period. An extended data collection period may be used to enable averaging or determination of variability. For example, measurement of heart activity such as heart rate or heart rate variability is performed over an extended data collection period. A typical data collection period for measuring heart activity is 1-2 minutes. It is realized that significant power savings may be achieved by increasing the time interval between sensor activation.

The method in FIG. 3 further comprises a step 35 that monitors the SPVs that are generated subsequent to step 34 for detection of a second switching point. When the second switching point is detected, step 36 causes the measurement rate to be increased, e.g. back to the first measurement rate. In FIG. 4, the second switching point is designated by SP2, and the increased measurement rate is represented as a time interval t1 between consecutive SPVs. As indicated in FIG. 4, the second switching point SP2 corresponds to a detection criterion that indicates that the user no longer has an elevated stress level.

As understood from the foregoing, the method 30 is designed for a specific length of the first time period ΔT1, which is selected to represent the underlying physiology of the stress system in humans. In one embodiment, first time period ΔT1 is in the range of 15-60 minutes, and preferably in the range of 20-45 minutes.

The time intervals t1, t2 (FIG. 4) may be given by the intended purpose and required performance of the stress monitoring, as well as considerations of power consumption and processing intensity. The time interval t2 may also be set in view of the second time period ΔT2, which may be in the range of 4-10 hours, to achieve an acceptable response time for detecting that the stress level in the user 1 is no longer elevated. In a non-limiting example, the time interval t1 may be in the range of 1-20 minutes, and preferably 5-15 minutes, and the time interval t2 (>t1) may be in the range of 2-60 minutes, and preferably 10-45 minutes.

FIG. 5 is a block diagram of an arrangement 50 for sensing physiological stress in a user. The arrangement comprises at least one sensor 10, which is associated with the user and operable to generate sensor data S. The arrangement 50 further comprises a computation module 51 which is operable to compute SPVs based on the sensor data S, and an evaluation module 52 which is configured to perform the method 30 of FIG. 3. As indicated in FIG. 5, the evaluation module 52 may be operable to generate a first control signal C1 for controlling the computation module 51 and a second control signal C2 for controlling the sensor 10. Thus, the computation module 51 may be configured to, in response to the first control signal C1, modify (reduce/increase) its SPV rate. Likewise, the sensor 10 may be configured to, in response to the second control signal C2, modify (reduce/increase) its duty cycle, i.e. the time interval between the data collection periods that produce the sensor data S (cf. FIG. 4). Depending on implementation, the control signals C1, C2 may either designate a rate (or time interval) to be used or merely indicate a switch between predefined rates (e.g. a default rate and a reduced rate). It is conceivable that the control signals C1, C2 are identical and generated as a single control signal by module 52. In some embodiments, control signal C1 may be omitted, e.g. if the module 51 is configured to compute each SPV whenever it has received sensor data S in the form of a fixed number of incoming data samples from the sensor 10. In such an embodiment, it may be sufficient to control, by control signal C2, the duty cycle of the sensor 10 to also change the SPV rate of the module 51. Conversely, in some embodiments, control signal C2 may be omitted, e.g. if the sensor 10 is configured to provide sensor data upon request by module 51. In such an embodiment, it may be sufficient to control, by control signal C1, the SPV rate of module 51 to also control the duty cycle of the sensor 10.

Generally, the connections between components in the arrangement 50 may be implemented by wired or wireless data transmission, based on any suitable communication protocol known in the art. Further, to the extent that components are arranged in a common device, the communication may involve operations of reading data from and writing data to a memory, as is well-known in the art.

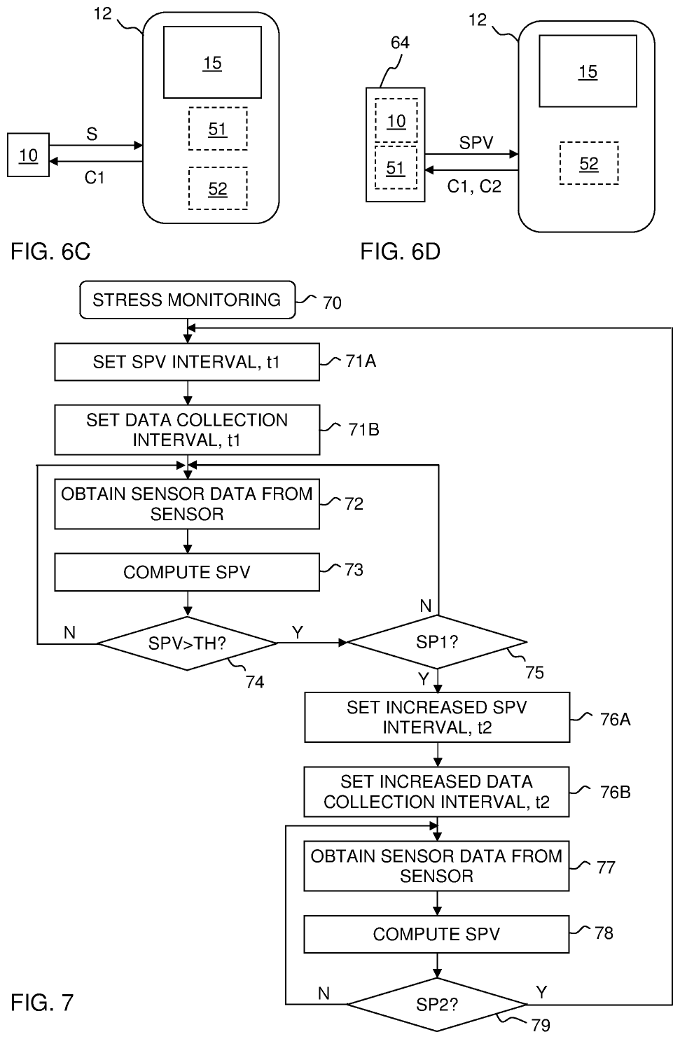

The arrangement 50 in FIG. 5 may be partitioned onto physical devices in different ways. Some non-limiting examples are illustrated in FIGS. 6A-6D.

In FIG. 6A, all components 10, 51, 52 are integrated into a single physical device, exemplified as a PED 12, which in this example also comprises a display 15 (cf. FIG. 1). The PED 12 in FIG. 6A may e.g. be a wearable computer which is attached to the body of the user, or a mobile phone. The mobile phone may be brought into engagement with the user to generate the sensor data S. For example, the mobile phone may command the user, by instructions on the display 15 or an audible signal, to bring the sensor 10 into engagement with the body.

Compared to FIG. 6A, the example in FIG. 6B differs by the computation module 51 being arranged in a separate computing device 60. In the illustrated example, the PED 12 is configured to communicate over a network 62 with the device 60, which may be a remote server such as a cloud server. The network 62 may comprise any combination of wide area and/or local area and/or personal area networks (WAN/LAN/PAN). As indicated in FIG. 6B, the PED 12 transmits sensor data S, generated by the sensor 10, to the server 60 which computes, by module 51, one or more SPVs and transmits the one or more SPVs to the PED 12. The evaluation module 52 in the PED 12 may control the SPV rate by transmission of control signal C1 to the server 60. In a variant, the device 60 may be a local device which may but need not be connected to the PED 12 by a network.

Compared to FIG. 6A, the example in FIG. 6C differs by the sensor 10 being separate from the PED 12. The sensor may e.g. be arranged on the body of the user and communicate sensor data S to the PED 12. The duty cycle of the sensor 10 may be controlled by transmission of control signal C1 from the PED 12 to the sensor 10.

Compared to FIG. 6C, the example in FIG. 6D differs by the sensor 10 and the computation module 51 being arranged in a sensing device 64 which is separate from the PED 12. The sensing device 64, which may but need not be attached to the body of the user, is configured to generate and output SPVs for receipt by the evaluation module 52 in the PED 12. The SPV rate of module 51 and the duty cycle of sensor 10 may be controlled by transmission of control signals C1, C2 from the PED 12 to the sensing device 64.

FIG. 7 is a flow chart of a more detailed example of a method 70 for sensing stress in a user. The method 70 will be presented with reference to the arrangement 50 in FIG. 5 and the data in FIG. 4. The method 70 may be performed by modules 51, 52 in combination. In step 71A, the time interval between SPVs to be generated by module 51 is set to a first value t1, e.g. by module 51 operating at a default setting or by module 52 generating the control signal C1. In step 71B, the data collection interval of the sensor 10 is also set to the first value t1, e.g. by the sensor 10 operating at a default setting or by module 52 generating the control signal C2. In step 72, module 51 obtains sensor data S from the sensor 10 in accordance with the time interval t1. In step 73, module 51 computes an SPV based on the sensor data S. In step 74, module 52 obtains the SPV and compares the SPV to the threshold TH. As long as the SPV falls below TH, the method repeatedly executes steps 72-73 to generate SPVs at the time interval t1, e.g. as illustrated in FIG. 4. If the SPV exceeds TH, step 74 proceeds to step 75, in which module 52 evaluates the SPVs for detection of the first switching point SP1, i.e. if the user has been stressed for more than ΔT1. In one implementation, step 75 detects SP1 when a predefined number of consecutive SPVs exceed TH, where the predefined number corresponds to an aggregated time period that is longer than ΔT1. The predefined number is at least two and may be larger to improve reliability of detection. In the example of FIG. 4, the predefined number is four. It should be realized that the time interval t1 is set with respect to ΔT1 so that a sufficient number of SPVs are obtained within ΔT1. If SP1 is not detected in step 75, the method returns to step 72. Otherwise, step 75 proceeds to step 76A, in which module 52 causes module 51 to generate SPVs at an increased time interval t2, e.g. by module 52 generating the control signal C1. In step 76B, module 52 causes the data collection interval to be increased to t2, e.g. by module 52 generating the control signal C2. Then, the method proceeds to repeatedly execute steps 77 and 78, until module 52 in step 79 detects the second switching point SP2. In step 77, module 51 obtains sensor data S from the sensor 10 in accordance with the time interval t2. In step 78, module 51 computes an SPV based on the sensor data S. Thereby, the arrangement 50 is operated to produce SPVs at a second, reduced rate. Step 79 may detect SP2 when one or more SPVs fall below the threshold TH. In the example of FIG. 4, SP2 is detected whenever a single SPV falls below TH. Step 79 may also comprise a time-out function that signals SP2 when a maximum time has elapsed since SP1. The maximum time is preferably equal to the second time period ΔT2 (FIG. 2). If SP2 is not detected in step 79, the method returns to step 77. Otherwise, step 79 proceeds to step 71A, whereby the arrangement 50 is again operated to produce SPVs at the first rate.

It should be noted that other detection criteria may be applied for detection of SP1, SP2 (steps 75,79). In one example, SP1 is detected whenever a predefined fraction of the SPVs generated during ΔT1 exceeds TH. In another example, SP2 is detected by comparing the SPVs to a second threshold that differs from TH.

It should also be noted that the time interval between consecutive SPVs may be increased in more than one step following detection of SP1.

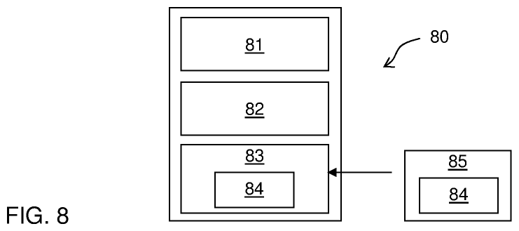

FIG. 8 is a diagrammatic representation of a machine 80 that may represent the PED 12. The machine 80 comprises a communication module 70 defining one or more interfaces for data communication in accordance with any suitable protocol or protocols. The machine 80 further comprises one or more processors 81, e.g. a central processing unit (CPU), a graphics processing unit (GPU), a digital signal processor (DSP), one or more application specific integrated circuits (ASICs), one or more radio-frequency integrated circuits (RFICs), a field programmable gate array (FPGA), or any combination thereof. The machine 80 further comprises system memory 82, which may include computer memory in the form of volatile and/or non-volatile memory such as read only memory (ROM), random access memory (RAM) and flash memory. The memory 82 may store computer instructions 83 (e.g. software or program code) for causing the machine 80 to perform any one of the methodologies discussed herein. The instructions 83 may be supplied to the machine 80 on a computer-readable medium 84, which may be a tangible (non-transitory) product (e.g. magnetic medium, optical medium, read-only memory, flash memory, digital tape, etc) or a propagating signal. When executed by the processor 81, the instructions 83 may cause the processor(s) 81 to perform any one of the methodologies discussed herein. In this context, it is to be understood that any of the modules 51, 52 described in the foregoing may be implemented by the processor(s) 81 executing the instructions 83. However, it is also conceivable that one or more of the modules 51, 52 are implemented solely by dedicated hardware in the machine 80.

1. A method of sensing stress in a user, said method comprising:
obtaining stress parameter values that represent a physiological stress level in the user and that are generated at a measurement rate based on sensor data from one or more sensors associated with the user;
monitoring the stress parameter values for detection of a switching point, wherein the switching point is detected when the stress parameter values indicate an elevated stress level in the user for a predefined time period; and
causing a reduction of the measurement rate upon detection of the switching point.

2. The method of claim 1, further comprising: causing the at least one sensor to be selectively activated and inactivated to generate the sensor data in coordination with the measurement rate.

3. The method of claim 1, wherein the predefined time period is in the range of 15-60 minutes, and preferably in the range of 20-45 minutes.

4. The method of claim 1, wherein the predefined time period corresponds to a time period in which the physiological stress in the user is at least partly controlled by adrenaline.

5. The method of claim 1, wherein the measurement rate is selected so that at least two, and preferably at least three, stress parameter values are obtained within the predefined time period.

6. The method of claim 1, wherein the switching point is detected when a predefined number of consecutive stress parameter values exceeds a stress threshold value.

7. The method of claim 1, further comprising, subsequent to the switching point, monitoring the stress parameter values for detection of a further switching pointer, and causing an increase in the measurement rate upon detection of the further switching point.

8. The method of claim 7, wherein the further switching point is detected when one or more of the stress parameter values fall below a stress threshold value within a further predefined time period subsequent to the switching point, or upon expiry of the further predefined time period.

9. The method of claim 8, wherein the further predefined time period is in the range of 4-10 hours.

10. The method of claim 1, further comprising: obtaining the sensor data from the one or more sensors and generating the stress parameter values based the sensor data.

11. The method of claim 1, wherein said sensor data represents one or more of heart activity, time interval between consecutive heart beats, heart rate, heart rate variability, skin conductivity, skin temperature, blood pressure, pupil diameter, eye movement, breathing pattern, blood oxygen saturation level, cardiac output, peripheral blood flow, muscle tension, and hormone content.

12. A computer-readable medium comprising computer instructions which, when executed by a processor, cause the processor- to perform the method of claim 1.

13. A device for sensing stress in a user, said device being configured to:
obtain stress parameter values that represent a physiological stress level in the user and are generated based on sensor data from one or more sensors associated with the user;
monitor the stress parameter values for detection of a switching point when the stress parameter values indicate an elevated stress level in the user for a predefined time period; and
cause a reduction of the measurement rate upon detection of the switching point.

14. The device of claim 13, further comprising at least one of said one or more sensors.

15. The device of claim 14, which is a mobile communication device, a portable electronic device or a wearable computer.

16. An arrangement for sensing stress in a user, comprising:
one or more sensors associated with the user;
a first module configured to generate, at a measurement rate and based on data sensor data generated by the one or more sensors, stress parameter values that represent physiological stress in the user; and
a second module configured to monitor the stress parameter values for detection of a predefined switching point when the stress parameter values indicate an elevated stress level in the user for a predefined time period, and to reduce the measurement rate upon detection of the predefined switching point.
